(19) United States
(12) Patent Application Publication
(10) Pub. No.: US 2026/0182851 A1
(43) Pub. Date: Jul. 2, 2026

(54) Implantable Pressure Sensors and Associated Methods

(71) Applicants: Emory University, Atlanta, GA (US); Children's Healthcare of Atlanta, Inc., Atlanta, GA (US); Georgia Tech Research Corporation, Atlanta, GA (US)

(72) Inventors: David Richard Myers, Atlanta, GA (US); Nicholas Au Yong, Atlanta, GA (US); Cecilia Alessandra Luna, Atlanta, GA (US); Brooks Lindsey, Atlanta, GA (US); Saeyoung Kim, Atlanta, GA (US); Bowen Jing, Atlanta, GA (US)

(21) Appl. No.: 19/118,727

(22) PCT Filed: Oct. 6, 2023
(86) PCT No.: PCT/US2023/034658

§ 371 Date: Apr. 4, 2025

Related U.S. Application Data
(60) Provisional application No. 63/414,204, filed on Oct. 7, 2022.

(51) Int. Cl. A61B 5/03; A61B 5/00; A61B 90/00

(52) U.S. Cl. CPC A61B 5/031; A61B 5/6864; A61B 90/39; A61B 2090/3925; A61B 2562/0247; A61B 2562/168

(57) ABSTRACT

The implanted pressure sensors can non-invasively and stably measure intracranial pressure using MRI-compatible materials. In some examples, the implanted pressure sensor can include a fluid reservoir filled with a fluid. The sensor may also include a gas chamber filled with a gas and a gas-fluid interface. The interface may include a channel. The channel may have a first end and a second end. The channel may have a pattern that includes a plurality of linear segments connected by a plurality of junction segments. The fluid reservoir may be in flow communication with the gas-fluid interface. The gas chamber may be in flow communication with the gas-fluid interface and spaced separate from the gas-fluid interface.

### CROSS-REFERENCE TO RELATED APPLICATIONS

This application claims the benefit of U.S. Provisional Application No. 63/414,204 filed Oct. 7, 2022. The entirety of this application is hereby incorporated by reference for all purposes.

### STATEMENT REGARDING FEDERALLY SPONSORED RESEARCH OR DEVELOPMENT

This invention was made with government support under EB031545 awarded by the National Institutes of Health. The government has certain rights in the invention.

### BACKGROUND

More than 1.3 million new patients are diagnosed each year with neurological conditions that may result in elevated intracranial pressure (ICP). Current techniques, such as using non-invasive MRI or CT imaging, rely on indirect indicators to measure intracranial pressure. Thus, these techniques have had limited ability to make accurate, stable pressure measurements and thus can contribute to poor clinical outcomes.

One solution that has been proposed has been implanted pressure sensors that can measure intracranial pressure. However, those proposed have had limited usefulness due to their drawbacks, such as size scales and the material constraints.

### SUMMARY

Thus, there is a need for implanted pressure sensor devices that can accurately and stably enable direct measurement of pressure within the skull.

Systems, devices, and methods disclosed herein relate generally to implantable pressure sensors devices that can be used with one or more ultrasound transducers to non-invasively measure pressure.

In some examples, the disclosed embodiments may include an implantable pressure sensor device. The device may include a fluid reservoir filled with a fluid. The device may also include a gas chamber filled with a gas. The device may further include a gas-fluid interface including a channel. The channel may have a first end and a second end. The channel may have a pattern. The pattern may include a plurality of linear segments connected by a plurality of junction segments. The fluid reservoir may be in flow communication with the gas-fluid interface. The gas chamber may be in flow communication with the gas-fluid interface and spaced separate from the gas-fluid interface.

In some examples, the disclosed embodiments may include the implantable pressure sensor device and a burr hole cover.

In some examples, the disclosed embodiments may include a method for noninvasively measuring pressure using an implanted sensor. The method may include acquiring an ultrasound image along one or more readout axes of the sensor using an ultrasound transducer. In some examples, the method may further include determining a quantitative measurement of pressure using the ultrasound image.

In some examples, the sensor may further include one or more sets of imaging markers. Each set of imaging markers may define a readout axis with respect to one or more segments of the gas-fluid interface. Each readout axis may correspond to a quantitative measurement of pressure. The one or more sets of imaging markers and the gas-fluid interface may define a read-out section. The one or more sets of imaging markers may be disposed on a boundary of the read-out section. The one or more sets of imaging markers may include a first set of imaging markers. The first set of imaging markers may define a first readout axis that traverses each linear segment of the readout section and having a first quantitative measurement of pressure. A cross-section of each linear segment along the first imaging axis may correspond to one increment of pressure

In some examples, the one or more sets of imaging markers may include a second set of imaging markers. The second set of imaging markers may define a second readout axis along one linear segment of the linear segments of the readout section. A cross-section of the one linear segment along the second imaging axis may correspond to a second quantitative measurement of pressure.

In some examples, the method may include acquiring an ultrasound image (e.g., first readout) along a first readout axis using the ultrasound transducer. In some examples, the quantitative measure of pressure may be determined using the image along the first readout axis. For example, the method may further include determining a quantitative measurement of pressure based on a number of visible dots.

In some examples, the method may further include acquiring an ultrasound image (e.g., a second readout) along one or more second readout axes using the ultrasound transducers. The method may further include determining a quantitative measurement of pressure for each second readout based on a length of a visible line with respect to a length of the corresponding linear segment.

In some examples, the one or more second readout axes may be based on the first readout along the first readout axis. In some examples, the one or more second readouts may be acquired for one or more of the linear segments along the first readout axis in the first readout. In some examples, the second readout may be acquired and/or the quantitative measurement may be determined along the respective second readout axis for 1) a linear segment that has an invisible or dark dot next to a linear segment that has a visible dot; and/or 2) the linear segment that has a visible dot. In this example, the quantitative measurement may be based on one or more of the second readouts.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 illustrates an example of a patient with an implantable pressure sensor system according to some embodiments.

FIG. 2A shows a top view of the implantable pressure sensor system of FIG. 1.

FIG. 2B shows an enlarged view of a portion of the implantable pressure sensor shown in FIG. 2A.

FIG. 3A shows an example of an implantable pressure sensor according to embodiments.

FIG. 3B shows a top view of the implantable pressure sensor of FIG. 3A.

FIG. 3C shows a side view of the implantable pressure sensor of FIG. 3A.

FIG. 4A shows a cross-sectional view of the implantable pressure sensor as shown in FIG. 3B.

FIG. 4B shows a cross-sectional view of the implantable pressure sensor as shown in FIG. 3C.

FIG. 5A shows another example of an implantable pressure sensor according to embodiments.

FIG. 5B shows a side view of the implantable pressure sensor of FIG. 5A.

FIG. 6A shows a cross-sectional view of the implantable pressure sensor as shown in FIG. 5A.

FIG. 6B shows a cross-sectional view of the implantable pressure sensor as shown in FIG. 6B.

FIG. 6C shows another view of the implantable pressure sensor shown in FIG. 6A.

FIG. 6C shows another side view of the implantable pressure sensor as shown in FIG. 6B.

FIG. 6D shows another side view of the implantable pressure sensor as shown in FIG. 6B.

FIG. 7 shows a cross-sectional view of the implantable sensor system according to embodiments.

FIG. 8 shows an example of a lookup table of the pressure measurements corresponding to a first readout axis according to embodiments.

FIG. 9A shows an example of a fluid/air in the gas-fluid interface of the implantable pressure sensors according to embodiments.

FIG. 9B shows an example of the diagnostic image of the fluid/gas interface as shown in FIG. 9A and associated visualization according to embodiments.

FIG. 10A shows another example of a fluid/air in the gas-fluid interface of the implantable pressure sensors according to embodiments.

FIG. 10B shows an example of the diagnostic image of the fluid/gas as shown in FIG. 10A according to embodiments.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to implantable pressure sensors that can directly measure and monitor intracranial pressure. In some examples, the implantable pressure sensors can be placed within a burr hole. In some examples, the implantable pressure sensors, according to embodiments, may be used with and/or integrated with a burr hole cover. In some examples, the implantable pressure sensor may be implanted so as to be at the skull level.

In some examples, the implantable pressure sensors can be configured to measure epidural, subdural, and/or intraventricular pressures. The pressure measured can depend on the space within the brain where the sensor is placed, for example, via a burr hole. For example, for intraventricular pressure measurements, the sensor may be inserted into the ventricular space, such as into the lateral ventricle; for subdural pressure measurements, the sensor may be inserted under the dural leaflets on top of the brain; and for epidural measurements, the sensor may be inserted in the space in the brain above the dura matter.

In some examples, the implantable pressure sensors may include a gas-fluid interface that is coupled to and in flow communication with (i) a fluid reservoir filled with a fluid and (ii) a gas chamber filled with a gas. A measurement of the fluid within the gas-fluid interface may correspond to quantitative measurement(s) of pressure, such as range of pressure values and/or a pressure value. When pressure increases, the pressure causes the fluid to move from the reservoir into the gas-fluid interface enabling an easy-to-read, quantitative pressure measurement along one or more readout axes defined in a readout section of the implantable pressure sensor using, for example, one or more ultrasound transducer(s).

In some examples, along at least one of the defined readout axes, the quantitative pressure measurement may be determined based on a total of increment(s) of pressure determined from the (linear) segments of the gas-fluid interface. In some examples, along the first readout axis, an increment of pressure may be visualized as a dot using an ultrasound transducer and may correspond to a cross-section of a linear segment of the gas-fluid interface. For example, along this readout axis, a visible or bright dot may correspond to a segment of the gas-fluid interface filled with air so may indicate no increment of pressure; and a dark or invisible dot may correspond to a segment filled with liquid so may indicate an increment of pressure. In this example, a series of bright dots may represent low pressure and a series of invisible dots may represent high pressure.

In some examples, the quantitative pressure measurement may be determined based on an increment(s) of pressure determined along at least one linear segment along at least another one of the defined readout axes. In some examples, along a second readout axis that is perpendicular to the first readout axis, an increment of pressure may be visualized as a line using an ultrasound transducer and may correspond to a length of a cross-section of that segment of the gas-fluid interface. For example, along the second readout axis, a long visible or bright line may correspond to a part of the segment of the gas-fluid interface filled with air so its length may indicate no increment(s) of pressure; and a dark or invisible line may correspond to a part of the segment filled with liquid so its length may indicate increment(s) of pressure. In this example, a long bright line may represent low pressure, and a short or no bright line may represent high pressure.

In some examples, the first readout along the first readout axis may be used to identify one or more linear segments from which a quantitative pressure measurement along the second readout axis should be determined. For example, a second readout along the second readout axis may be acquired for: 1) a linear segment that has an invisible or dark dot (directly) next to a linear segment that has a visible dot; and/or 2) the linear segment that has a visible dot, using one or more ultrasound transducers. In this example, the quantitative measurement may be based on the increments for: 1) a linear segment that has an invisible or dark dot (directly) next to a linear segment that has a visible dot; and/or 2) the linear segment that has a visible dot, using one or more ultrasound transducers.

Intracranial pressure measurements using the sensors, according to the embodiments, can be performed in a clinician's office without requiring highly-skilled care and resources. By enabling a stable, accurate pressure measurement, patient outcomes can be improved while reducing the use of healthcare resources.

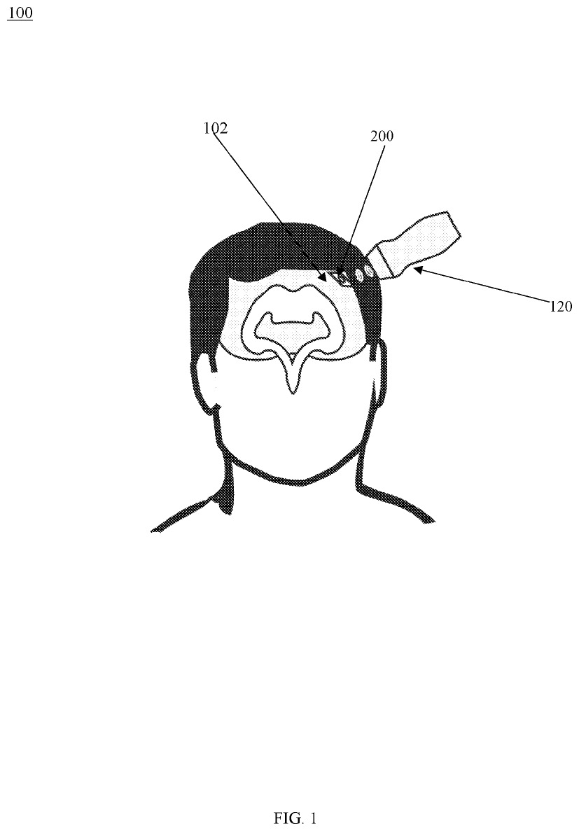

FIGS. 1-7 show examples of implantable pressure sensors according to some embodiments. It will be understood that the implantable pressure sensors are not limited to the configuration and/or combination of the fluid reservoir, gas chamber, and gas-fluid interface, as shown and described with respect to the figures. The implantable pressure sensors may include any combination of the embodiments and/or alternative embodiments of the fluid reservoir(s), gas chamber(s), and gas-fluid interface(s) as described.

FIG. 1 shows an example 100 of a patient with an implantable pressure sensor 200 system according to some embodiments. In this example, the pressure sensor system 200 may be implanted into the ventricular space. As shown, an ultrasound transducer 120 may be used to visualize the gas-fluid interface and thus report the pressure measurement.

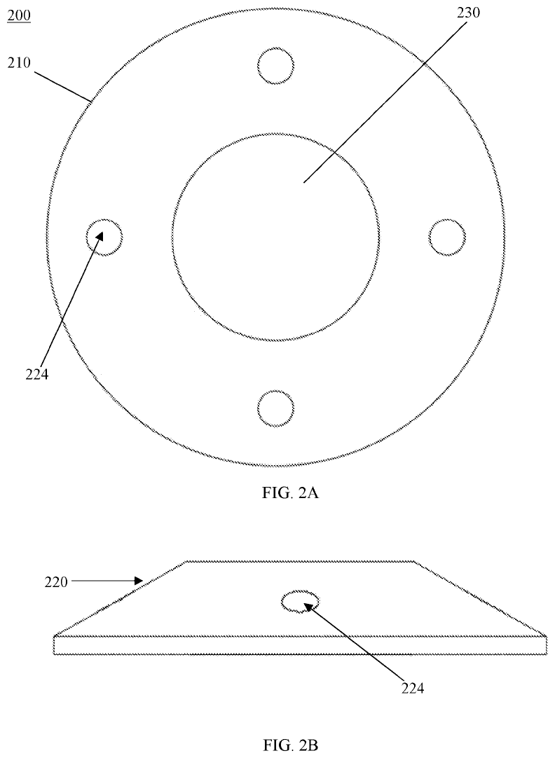

FIGS. 2A and 2B show an example of an implantable pressure system 200. In some examples, the system 200 may include a burr hole cover 210 in which an implantable pressure sensor 230 may be disposed and/or integrated. As shown, the burr hole cover 210 may include a burr hole base 220 and cap (not shown) that covers the sensor 230. The base 220 may include openings 224 in which screws may be positioned to secure the burr hole base 220 to the burr hole and scalp of the patient. It will be understood that the burr hole cover 210 is not limited to the type shown and may include any available burr hole cover.

In some examples, the pressure sensor 230 may be a closed-system device. In some examples, the pressure sensor 230 may include a gas-fluid interface. In some examples, the gas-fluid interface may include a channel. The channel may be disposed in a pattern. In some examples, the pattern may include a plurality of linear segments connected by a plurality of junction segments. In some examples, the junction segments may have a curved shape. For example, the channel of the gas-fluid interface may have a serpentine shape. In other examples, the channel may have a different shape.

In some examples, the pressure sensor 230 may include a fluid reservoir that is coupled in flow communication with one end of the gas-fluid interface and a gas chamber that is coupled in flow communication with the other end of the gas-fluid interface. In some examples, the fluid reservoir may include one or more surfaces configured to deflect, for example, when pressures at the insertion point increase, causing the fluid to move from the fluid reservoir to the gas-fluid interface. In some embodiments, the one or more surfaces may be configured to deflect. The one or more surfaces may be made of one or more materials including but not limited to polyurethane, silicone, cold form foil (thin metals with plastic coverings), polyetheretherketone (PEEK), polytetrafluoroethylene (PTFE), thin glass, titanium, among others, or a combination thereof. In some examples, the fluid reservoir may be a balloon. In other examples, the fluid reservoir may include at least one surface (e.g., membrane) configured to deflect. In some examples, the one or more surfaces configured to deflect may be smooth and/or textured.

In some examples, the fluid reservoir may be filled with an amount of fluid. The fluid may be any biocompatible fluid, including but not limited to saline, water, among others, or a combination thereof. The fluid reservoir may be disposed separate from the gas-fluid interface. For example, the fluid reservoir may be separated by a conduit (such as a channel) and may be disposed in a different layer and/or plane so as to be below the gas-fluid interface when inserted into a burr hole.

In some examples, the gas chamber may be filled with an amount of gas. The gas may be any biocompatible gas, including but limited to air, nitrogen, among others, or any combination thereof. The gas chamber may be disposed separate from the gas-fluid interface. In some examples, the gas chamber may be distinct from the gas-fluid interface. For example, the gas chamber may be at least separated from the gas-fluid interface by a conduit (such as a channel). In some examples, the gas chamber may be disposed above the gas-fluid interface when inserted into a burr hole. This way, the gas-fluid interface may be disposed between the fluid reservoir and the gas chamber with regards to the flow communication.

In some examples, the pressure sensor 230 may be made of one or more MRI-compatible materials, including but not limited to polydimethylsiloxane (PDMS), other elastomers, glass, plastics, composites, other MRI-compatible diamagnetic metals, or any combination thereof. In some examples, the pressure sensor 230 may be coated and/or encapsulated with a biocompatible material such as parylene, polyurethane, PEEK, PTFE, thin metal coatings, glass, drug-eluting polymers, among others, or any combination thereof.

In some examples, the pressure sensor 230 may include one or more sets of imaging markers disposed in the same plane as the gas-fluid interface. The one or more imaging markers may be fiducial markers that can be visualized by one or more imaging modalities, such as one or more ultrasound transducers. In some examples, the imaging markers may have any shape. The shape may include but is not limited to a circle, square, etc. In some examples, the imaging markers may be a square-or circular-shaped encased-chamber filled with gas, such as air. For example, the imaging markers may be made within the same layer and material as the sensor. In other examples, the imaging markers may be made of a different material.

In some examples, each set of imaging markers may define a readout axis with respect to one or more segments of the gas-fluid interface. This way, each set of the imaging markers may identify an imaging plane for the ultrasound transducer to visualize a pressure measurement. By way of example, the one or more sets of imaging markers may include one or more (first) sets of imaging markers disposed so that the corresponding (first) readout axes transverse each linear segment. The one or more first readout axes may be disposed anywhere along the length of the linear segments between the corresponding junction segments. In some examples, the readout for each first readout axis may be visualized as one or more dots when gas is present in the respective segment of the gas-fluid interface. Each dot may correspond to a cross-section of a linear segment, and each cross-section/dot may correspond to an increment of pressure (e.g., a pressure value or a range of values). In this example, the pressure measurement may be determined by counting the dots.

In some examples, the one or more sets of imaging markers may include one or more (second) sets of imaging markers disposed so that the corresponding readout axes are along a length of linear segment(s). In this example, the readout may be visualized as a line when gas is present in that segment of the gas-fluid interface. The line/length of cross-section along the linear segment may correspond to a pressure measurement.

By way of example, pressure measurement and/or an increment of pressure may correspond to a pressure value and/or range of pressure values.

In some examples, the pressure sensor 230 may be calibrated for specific increments of pressure. For example, the dimensions and/or configuration and/or pattern of the channel of the gas-fluid interface, the amount of fluid and/or gas stored within the sensor, among others, may vary depending on the desired pressure increment measurements. By way of example, the number of linear segments, the length of linear segments, total length of the channel, among others, may vary depending on the desired pressure increment measurements.

In some examples, the diameter of the channel of the gas-fluid interface may be equal to or greater than about 110 μm, and the channel center-to-center spacing of the gas-fluid interface may be about 1 mm. The dimensions are not limited to 110 μm/1 mm, and the dimensions of the channel of the gas-fluid interface may be different, such as smaller or larger than 110 μm and/or 1 mm. In some examples, the gas chamber may have a volume of about 15 mm³. The dimensions are not limited to 15 mm3, and the dimensions of the channel of the gas-fluid interface may be different, such as smaller or larger than 15 mm³.

In some examples, the one or more sets of imaging markers and the gas-fluid interface bounded by the imaging markers may define a read-out section. In this example, the one or more sets of imaging markers may be disposed on a boundary of the readout section.

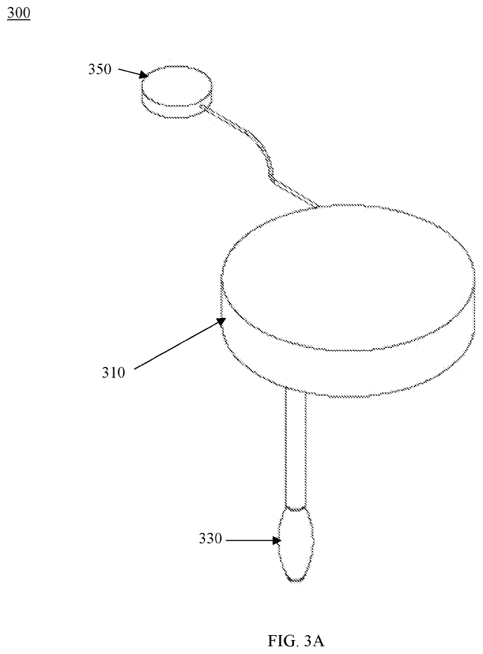

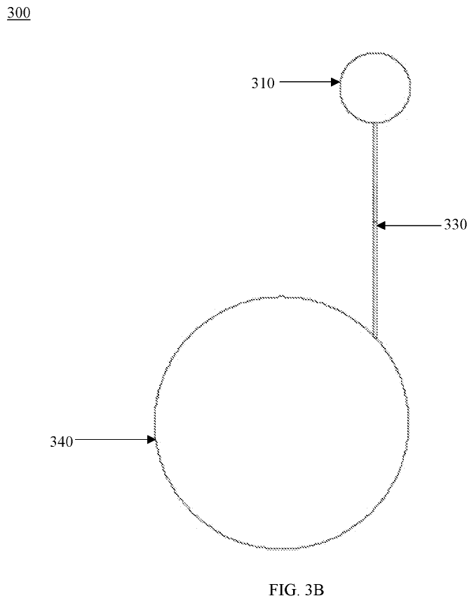

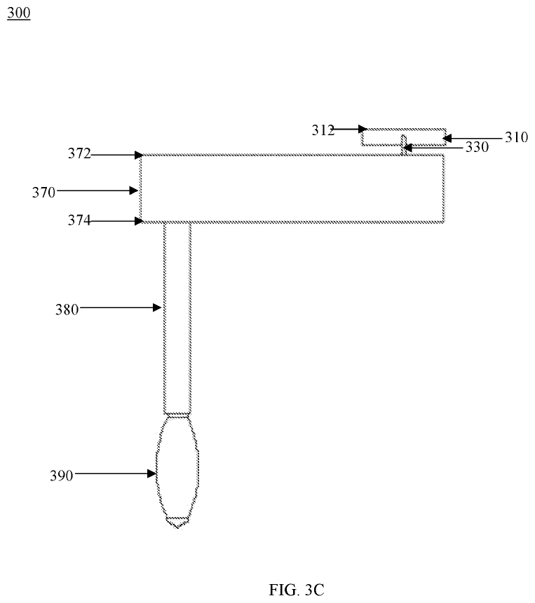

In some examples, FIGS. 3A-4B and FIGS. 5A-6D show examples of pressure sensor 230 according to some embodiments.

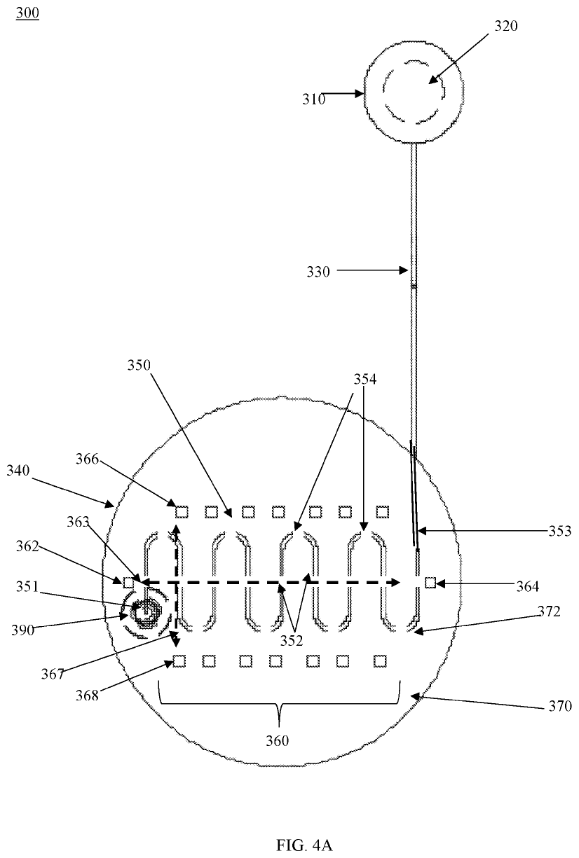

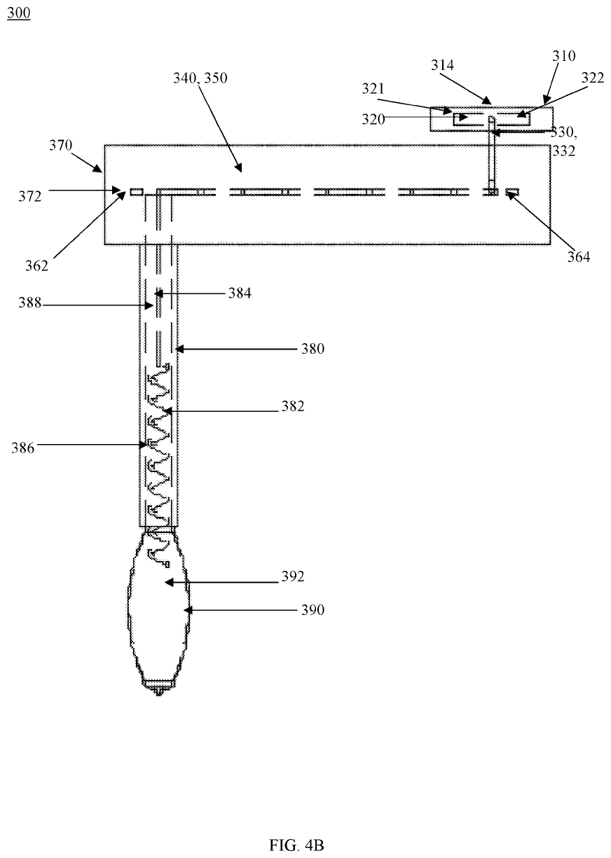

FIGS. 3A-4B show an example of a pressure sensor 300 according to some embodiments. In some examples, the pressure sensor 300 may include a fluid reservoir 390 that is configured to be separately disposed at a target region for which pressure is to be measured by the sensor 300. In some examples, the target region may correspond to the type of pressure to be measured. The target region may include but is not limited to within the ventricle system, epidural space (above the dura), subdural space (e.g., right cerebral hemisphere), other regions of the brain, among others, or a combination thereof.

In this example, the fluid reservoir 390 may include one or more surfaces configured to deflect upon a rise in pressure at the target region. For example, in this example, at least the circumferential surface of fluid reservoir 380 may be configured to deflect. In some examples, the fluid reservoir 390 may be a balloon. The fluid reservoir 390 may include a fluid 392 (e.g., an amount of any biocompatible fluid such as water).

In some examples, the pressure sensor 300 may include a gas-fluid interface 340 that is coupled in flow communication with the fluid reservoir 390, for example, via conduit 380. In this example, the gas-fluid interface 340 may include a channel 350. The channel 350 may include a first end 351, a second end 353, and length therebetween. The channel 350 may be disposed in a pattern along its length. The pattern may include a plurality of linear segments 352 connected by a plurality of junction segments 354. In some examples, the junction segments 354 may be curved so that the pattern has a serpentine shape. The channel 350 of the gas-fluid interface 340 may include any number of segments and is not limited to the nine linear segments 352 and eight junction segments 354 shown in FIG. 4A. For example, the gas-fluid interface 340 may include more or less linear and/or junction segments.

In some examples, the sensor 300 may include one or more sets of imaging markers 360 disposed in the same plane as the gas-fluid interface 340. In some examples, the imaging markers 360 may have any shape (e.g., circular) and is not limited to the square shape shown. In this example, each imaging marker 360 may be a chamber formed within one or more of the materials of the gas-fluid interface 340 and filled with gas, such as air.

In some examples, each set of imaging markers 360 may define a readout axis with respect to one or more segments of the gas-fluid interface. This way, each set of the imaging markers may identify an imaging plane for the ultrasound transducer to visualize a pressure measurement.

In some examples, the one or more sets of imaging markers 360 may include one or more (first) sets of imaging markers disposed so that the corresponding first readout axis transverse each linear segment. For example, the one or more sets of imaging markers 360 may include at least one (first) set of imaging markers 362, 364 that define a first readout axis 363 (identified as a dashed line for illustration purposes only) that transverse each linear segment. In some examples, the first readout axis 363 may be in about the center of the linear segments 352 with respect to the junction segments 354, for example, as shown in FIG. 4A. The first readout axis 363 and/or one or more additional first readout axes may be disposed at a different position across the linear segments 352 with respect to the junction segments 354. For example, the one or more first readout axes may be disposed across the linear segments 352 closer to one of the junction segments 354.

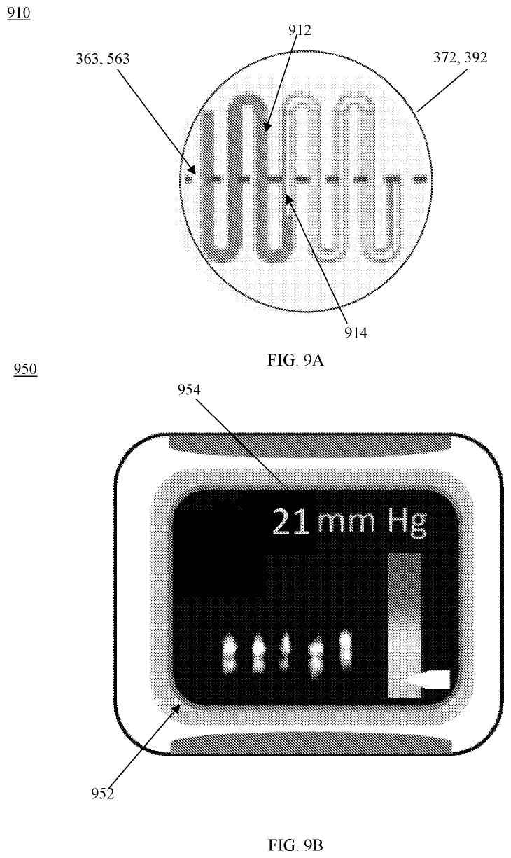

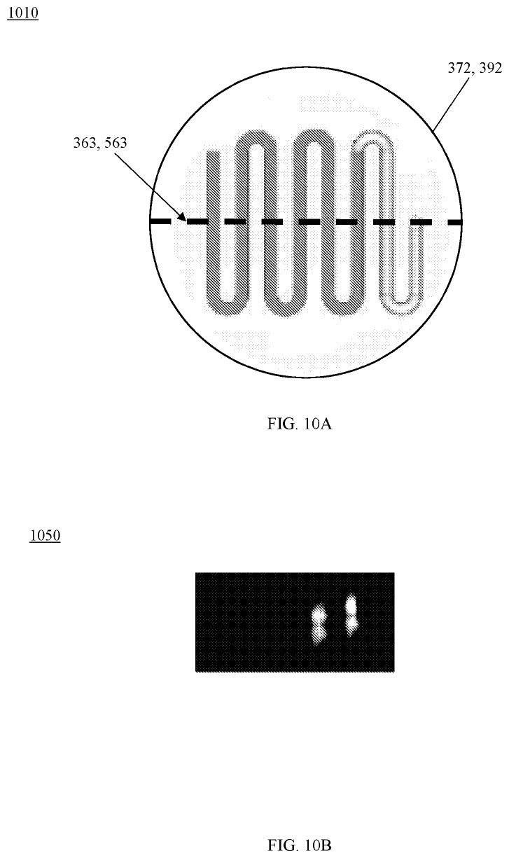

For example, when interrogated by one or more ultrasound transducers along the first readout axis 363, the (first) readout may be visualized as one or more dots when gas is present in the respective segment of the gas-fluid interface channel 350, for example, as shown in FIGS. 9A and 10A. Each dot may correspond to a cross-section of a linear segment and each cross-section/dot may correspond to an increment of pressure. In this example, the measured pressure along the first readout axis 363 may be determined by counting the dots.

In some examples, the one or more sets of imaging markers 360 may optionally also include one or more (second) sets of imaging markers 366, 368 disposed so that the corresponding second readout axes 367 (identified as a dashed line for illustration purposes only) defined by the second set of imaging markers 366, 368 are along a length of one or more linear segment(s). As shown, the second readout axes 367 may be perpendicular to the first readout axes 363. If the sensor 300 includes one or more second sets of imaging markers, there may be any number of sets and is not limited to the seven sets and seven second readout axes shown. In this example, when interrogated by an ultrasound transducer along one of the second readout axes 367, the (second) readout may be visualized as a line when gas is present in the gas-fluid interface channel 350 along that segment. The line/length of linear segment may correspond to an increment of pressure.

In some examples, the imaging markers 360 and the gas-fluid interface 340 bounded by the imaging markers 360 may define a readout section 372. In this example, the one or more sets of imaging markers 360 may be disposed on a boundary of the readout section 372.

In some examples, the sensor 300 may include a body 370. The body 370 may include the gas-fluid interface 340. In some examples, the readout section 372, the gas-fluid interface 340, and the markers 360 may be disposed in the same plane and/or layer of the body 370.

In some examples, the pressure sensor 300 may include a gas chamber 320 that is coupled in flow communication with the channel 350, for example, via conduit 330. The gas chamber 320 may include a gas 322 (e.g., an amount of any biocompatible gas such as air). In some examples, the gas chamber 320 may include one or more deflectable surfaces. For example, the gas chamber 322 may include a surface 321 that is configured to be deflectable. In some examples, the surface 321 may be made of one or more materials having deflectable properties, including but not limited to polyurethane, silicone, cold form foil (thin metals with plastic coverings), PEEK, PTFE, thin glass, titanium, among others, or a combination thereof.

In some examples, the sensor 300 may include a gas chamber body 310. In some examples, the gas chamber body 310 may include an actuating member 314 (e.g., a button) disposed on the top surface 312 of the body 310 and configured to be actuated by (e.g., pressed) a clinician's finger or a tool to engage the surface 321 of the gas chamber 320. When the actuating member 314 is actuated, it can cause the surface 321 to push against the chamber 320, causing the gas 322 from the chamber 320 to flow to the gas-fluid interface 340 via the conduit 330. In some examples, the body 310 may be used to test the accuracy of the pressure sensor 300.

In some examples, the gas chamber 320 may be coupled in flow communication to the second end 353 of the channel 350 of the gas-fluid interface 340 via the conduit 330, and the fluid reservoir 390 may be coupled in flow communication to the first end 351 of the channel 350 of the gas-fluid interface 340 via the conduit 380. In some examples, the conduit 330 may include a channel 332, and the conduit 380 may include a channel 382. As shown in FIG. 4B, in some examples, the channel 332 may be elongated along its length, and the channel 382 may include a coiled section 386 and an elongated section 384 along its length. In some examples, the channels 332 and/or 382 may have a different configuration.

In some examples, the conduits 330 and 380 may be made of an MRI-compatible material. In some examples, the conduits 330 and 380 may be made of the same materials as the body 370, such as PDMS, glass, polyurethane, PEEK, PTFE, composites of metal with plastic, among others, or a combination thereof.

In some examples, the sensor 300 may include a valve disposed along one or more conduits. In some examples, the sensor 300 may include a valve 388 disposed along the length of the channel 382. In some examples, the valve 388 may be disposed along the elongated section 384. In some examples, the valve 388 may be a two-way valve configured to retain the fluid 392 in the fluid chamber 390 during surgical placement of the sensor 300, and be opened, for example, by engaging a releasing member (e.g., loosening a screw, removing a member, etc.), once the placement is completed. In this example, the fluid 392 may remain in the fluid chamber 390 and separate from the gas 322, and the valve 388 may prevent them from inadvertently mixing during implantation of the sensor 300.

In some examples, the gas-fluid interface 340/the body 370 may be disposed between the fluid reservoir 390 and the gas chamber 320/gas chamber body 310 so that (i) the fluid reservoir 390 is below the gas-fluid interface 340 and (ii) the gas chamber 320 is above the gas-fluid interface.

In some examples, the pressure sensor 300 may be inserted using available neurological techniques and tools. By way of example, when using a type of burr hole cover similar to the cover 210, after a burr hole of a patient has been created and the target region (e.g., desired measurement site, which can depend on the pressure to be measured, such as subdural, lateral ventricle, dura space within the brain, etc.) has been incised using standard neurosurgical techniques and tools, a burr hole base may be secured to the skull. For example, using standard neurological techniques and tools (e.g., peel-away sheath cannula), the pressure sensor 300 may be then placed within the burr hole and the burr hole base 220, so that the fluid reservoir 390 may be disposed within the target region of the brain and the base 220 of the burr hole cover 210 and the cap may be attached. In some examples, the gas chamber body 310 may be disposed under the cap, on the cap/cover, external to the cover (e.g., affixed next to the cover between the skull/skin with a fastener member, such as a screw), among others, or any combination thereof. In other examples, other available neurological techniques and tools may be used, including different burr hole covers.

After the pressure sensor 300 is inserted, the sensor 300 may be tested to confirm the sensor's accuracy and operation status. For example, the failure modes of the sensor 300 may include but are not limited to: 1) a loss of mechanical sensitivity from biofilm formation; 2) a loss of mechanical sensitivity from a fluid leak; 3) a loss of accuracy from drift, which would occur from a change in the volume of gas; 4) among others; 5) or any combination thereof. To test for these failure modes, a clinician can use the pressure sensor 300 in reverse by actuating (e.g., pressing) the actuating member 314 disposed above the skull using a finger or other tool, for example, for a set period of time and then release. The pressure can be continuously recorded immediately before, during, and after the actuation of the actuating member 314. The recorded pressure can be used to determine the sensor's operating status, for example, whether the sensor is in normal mode or a failure mode. The pressure recorded before may be used to determine whether the pressure during and after release is within normal operating status. For example, the pressure measured during the actuation of the actuating member 314 should decrease within a threshold and increase after the actuating member is released within a threshold. If the measured pressure during these phases is outside at least one of the thresholds associated with the phases, the sensor 300 may be in a failure mode. If the measured pressure during these phases is within the threshold for each phase, the pressure sensor 300 may be determined to be ready to be used.

In some examples, the gas chamber 320 may be included within the body 370. In this example, the sensor 300 may omit the gas chamber body 310 (and actuating member 314) and may include the conduit 330 and gas chamber 320 within the body 370, for example, as discussed with respect to pressure sensor 500. In this example, the gas chamber 320 may omit the one or more deflectable surfaces.

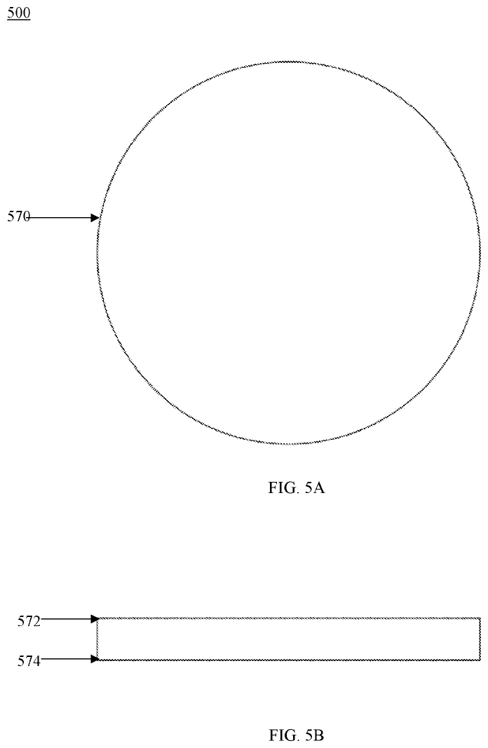

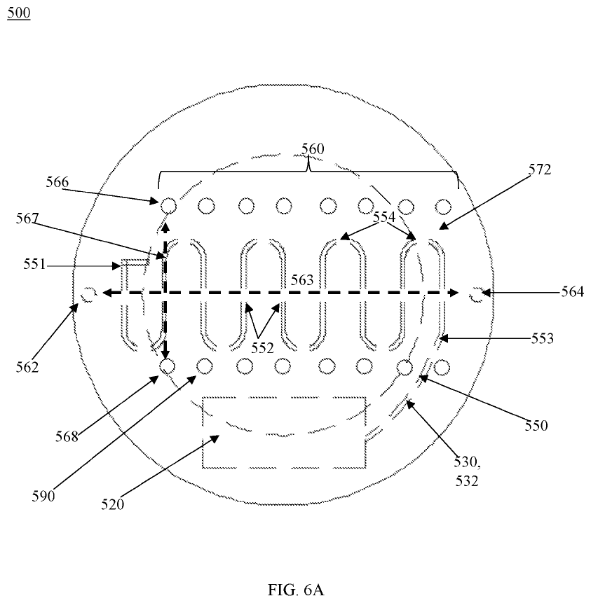

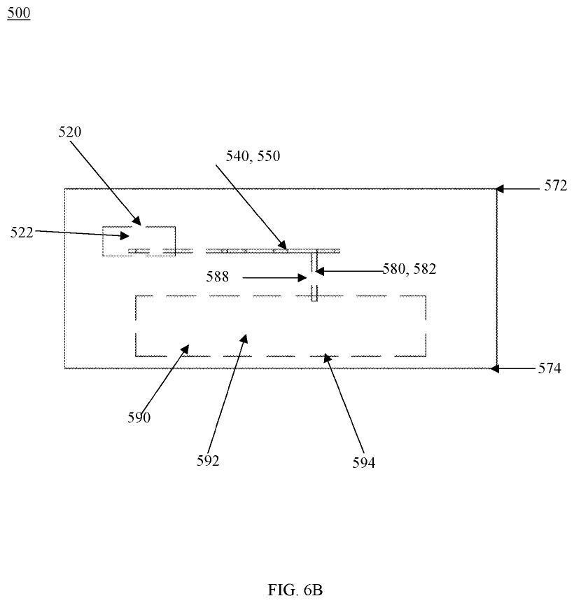

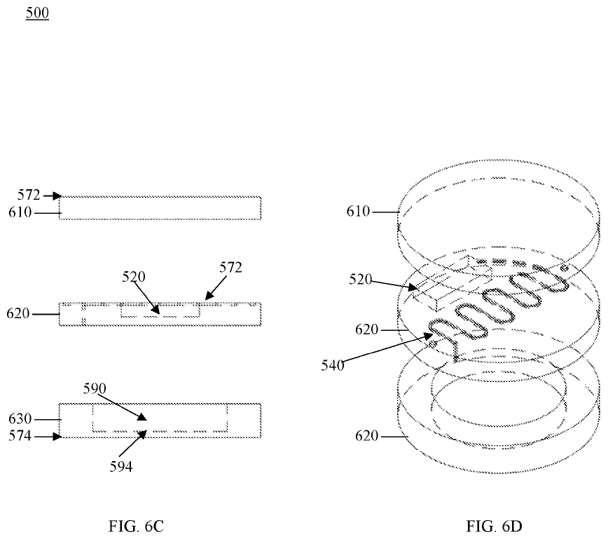

FIGS. 5A-6D show another example of a pressure sensor (500) according to some embodiments. In some examples, the pressure sensor 500 may include a fluid reservoir 590. In this example, the fluid reservoir 590 may include one or more surfaces configured to deflect upon a rise in pressure at the target region. For example, at least one surface 594 of the fluid reservoir 580 may be configured to deflect, for example, when pressure rises at the target region. The fluid reservoir 590 may include a fluid 592 (e.g., an amount of any biocompatible fluid such as water).

In some examples, the pressure sensor 500 may include a gas-fluid interface 540 that is coupled in flow communication with the fluid reservoir 590, for example, via conduit 580. In this example, the gas-fluid interface 540 may include a channel 550. The channel 550 may include a first end 551, a second end 553, and length therebetween. Like the channel 350, the channel 550 may be disposed in a pattern along its length. The pattern may include a plurality of linear segments 552 connected by a plurality of junction segments 554. In some examples, the junction segments 554 may be curved so that the pattern has a serpentine shape. The channel 550 of the gas-fluid interface 540 may include any number of segments and is not limited to the nine linear segments 552 and eight junction segments 554 shown in FIG. 5A. For example, the gas-fluid interface 540 may include more or less linear and/or junction segments.

In some examples, like the sensor 300, the sensor 500 may include one or more sets of imaging markers 560 disposed in the same plane as the gas-fluid interface 540. In some examples, the imaging markers 560 may have any shape (e.g., square) and are not limited to the circular shape shown. In this example, the imaging markers 560 may be a chamber formed within one or more of the materials of the gas-fluid interface 540 (e.g., PDMS) and filled with gas, such as air.

In some examples, each set of imaging markers 560 may define a readout axis with respect to one or more segments of the gas-fluid interface 540. This way, each set of the imaging markers 560 may identify an imaging plane for the ultrasound transducer(s) to visualize a pressure measurement.

In some examples, like the sensor 300, the one or more sets of imaging markers 560 may include one or more (first) sets of imaging markers disposed so that the corresponding first readout axes transverse each linear segment. For example, the one or more sets of imaging markers 360 may include at least one (first) set of imaging markers 562, 564 that define a first readout axis 563 (identified as a dashed line for illustration purposes only) that transverse each linear segment. In some examples, the first readout axis 563 may be in about the center of the linear segments 552 with respect to the junction segments 554, for example, as shown in FIG. 6A. The first readout axis 563 and/or one or more additional first readout axes may be disposed at a different position across the linear segments 552 with respect to the junction segments 554. For example, the one or more first readout axes may be disposed across the linear segments 552 closer to one of the junction segments 554.

For example, when interrogated by one or more ultrasound transducers along the first readout axis 563, the (first) readout may be visualized as one or more dots when gas is present in the respective segment of the gas-fluid interface channel 550, for example, as shown in FIGS. 9A and 10A. Each dot may correspond to a cross-section of a linear segment and each cross-section/dot may correspond to an increment of pressure. In this example, the measured pressure along the first readout axis 563 may be determined by counting the dots.

In some examples, like the sensor 300, the one or more sets of imaging markers 560 may optionally also include one or more (second) sets of imaging markers 566, 568 disposed so that the corresponding (second) readout axes 567 (identified as a dashed line for illustration purposes only) defined by the second set 566, 568 are along linear segment(s). As shown, the second readout axes 367 may be perpendicular to the first readout axes 363. If the sensor 500 includes one or more second sets of imaging markers, there may be any number of sets and is not limited to the seven sets and seven second readout axes shown. In this example, like the sensor 300, when interrogated by one or more ultrasound transducers along one of the second readout axes 567, the (second) readout may be visualized as a line when gas is present in the gas-fluid interface channel 550 along that segment. The line/length of linear segment may correspond to an increment of pressure.

In some examples, like the sensor 300, the imaging markers 560 and the gas-fluid interface 540 bounded by the imaging markers 560 may define a readout section 572. In this example, the one or more sets of imaging markers 560 may be disposed on a boundary of the readout section 572.

In some examples, like the sensor 300, the sensor 500 may include a body 570. The body 570 may include the gas-fluid interface 540. In some examples, the readout section 572, the gas-fluid interface 540, the markers 560 may be disposed in the same plane and/or layer of the body 570.

In some examples, the pressure sensor 500 may include a gas chamber 520 that is coupled in flow communication with the channel 550, for example, via conduit 330. The gas chamber 520 may include a gas 522 (e.g., an amount of any biocompatible gas such as air).

In some examples, the gas chamber 520 may be coupled in flow communication to the second end 553 of the channel 550 of the gas-fluid interface 540 via the conduit 530, and the fluid reservoir 590 may be coupled in flow communication to the first end 551 of the channel 550 of the gas-fluid interface 540 via the conduit 580. In some examples, the conduit 530 may include a channel 532 and the conduit 580 may include a channel 582.

In some examples, the conduits 530 and 580 may be made of an MRI-compatible material. In some examples, the conduits 530 and 580 may be made of the same materials as the body 570, such as PDMS, glass, polyurethane, PEEK, PTFE, composites of metal with plastic, among others, or a combination thereof.

In some examples, the sensor 500 may include a valve 588 disposed along the length of the channel 582. In some examples, the valve 588 may be a two-way valve configured to retain the fluid 592 in the fluid chamber 590 during surgical placement of the sensor 300, and be opened, for example, by engaging a releasing member (e.g., loosening a screw, removing a member, etc.), once the placement is completed. In this example, the fluid 592 may remain in the fluid chamber 590 and separate from the gas 522, and the valve 588 may prevent them from inadvertently mixing during implantation of the sensor 500.

In some examples, the gas-fluid interface 540 may be disposed between the fluid reservoir 590 and the gas chamber 520 in flow communication. In some examples, the fluid reservoir 590 may be disposed below the gas-fluid interface 540.

In some examples, the sensor 500 may include one or more layers. For example, as shown in FIGS. 6C and 6D, the sensor 500 may include a first layer 610, a second layer 620, and a third layer 630. The second layer 620 may be disposed in between the first layer 610 and the third layer 630. In some examples, the fluid reservoir 590 may be disposed in layer 630. In some examples, the readout section 572 (e.g., interface 540 and markers 560) and the gas chamber 520 may be disposed in the second layer 620. In this example, the readout section 672 may be disposed so that is separated from the chamber 520 by the conduit 520 so that they are distinct components. For example, the readout section 572 may be disposed in between the gas chamber 520 and the reservoir 590 with regards to flow communication, as shown in the side view shown in FIG. 6B. In other examples, the gas chamber 520 may be disposed in a different layer.

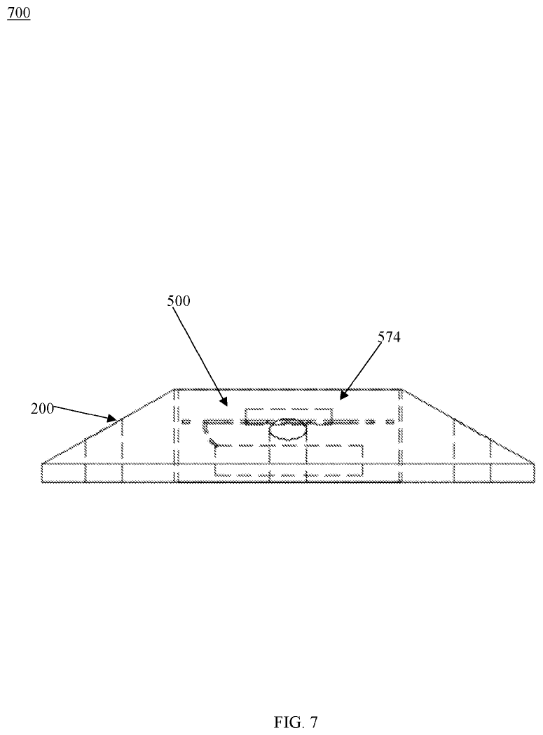

In some examples, the pressure sensor 500 may be inserted using available neurological techniques and tools. By way of example, when using a type of burr hole cover similar to the cover 210, after a burr hole of a patient has been created and the target region (e.g., desired measurement site, which can depend on the pressure to be measured, such as subdural, lateral ventricle, dura space within the brain etc.) has been incised using standard neurosurgical techniques and tools, a burr hole base may be secured to the skull so as to surround the burr hole. For example, using standard neurological techniques and tools (e.g., peel-away sheath cannula), the pressure sensor 500 may be placed within the burr hole and the base 220 so that the fluid reservoir 590 may be disposed against the target region of the brain. In some examples, the burr hole cap may then be placed on the burr hole base 220 over the pressure sensor 500. In some examples, when the sensor 500 is placed within the burr hole base 220, there may be a space above the top surface 574 of the sensor 500 in the center of the burr hole base 220, for example, as shown in FIG. 7. In other examples, other available neurological techniques and tools and/or burr hole covers may be used, including different burr hole covers.

In some examples, to obtain measurements from an implanted sensor 300, 500, a clinician may place one or more ultrasound transducers at ninety degrees with respect to one or more readout axes. Each ultrasound transducer may acquire an image (e.g., a readout) and transmit it wirelessly or via a wired system to a system for generation and display for display of the image and the pressure measurement. FIG. 9B shows an example of the display. In some examples, the pressure sensor 300, 500 may be part of a system that includes the burr hole cover. In some examples, the system may also include ultrasound transducer(s) and analysis/visualization system.

In some examples, a pressure measurement may be determined along one of the readout axes, such as the first readout axis. In some examples, the first readout along the first readout axis may determine whether a second readout should also be acquired. In some examples, the first readout may determine which linear segments for which, if any, a (second) readout along the second readout axis should be acquired. For example, a second readout may be acquired for 1) a linear segment that has an invisible or dark dot next to a linear segment that has a visible dot; and/or 2) the linear segment that has a visible dot in the in the first readout.

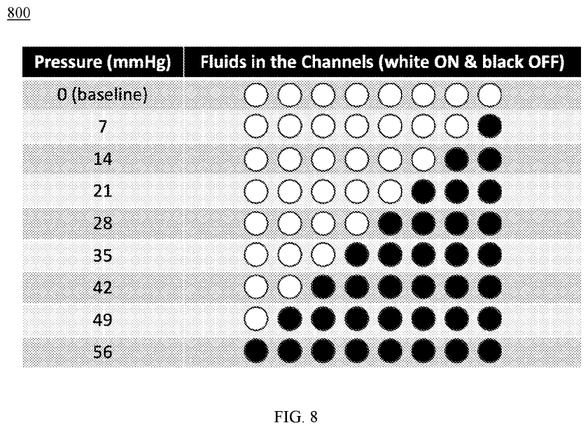

FIGS. 8-10A show examples of pressure measurements based on exemplary readouts of the sensors 300 and 500. FIG. 8 shows an example of a look-up table of pressure increments corresponding to gas/fluid disposed in the linear segments along the first readout axis 363, 563. In this example, each linear cross-section may correspond to an increment of about 7 mm HG. These increments are for illustration purposes and is not limited to the discrete increments listed. In some examples, a sensor, according to embodiments, may have different increments, such as different discrete values and/or range of values, of pressure associated with a first readout axis.

To obtain these measurements, a clinician can acquire an image (also referred to as “first readout”) along the first readout axis 363, 563 of an implanted sensor 300, 500 by positioning an ultrasound transducer ninety degrees aligned with the first readout axis 363, 563. After the image is acquired, it may be sent to a system for analysis and/or display.

FIG. 9A shows an example 910 of the gas-fluid changes in the gas-fluid interface 340, 540 within the readout section 372, 392 based on the state of a target region of a patient. In this example, the membrane/surface of the fluid reservoir 390, 592 caused the fluid to move through three linear and junction segments of the channel 350, 950, as shown in FIG. 9A. FIG. 9B shows an example of a display 950 of the results of the ultrasound scan of the example 910 along the first readout axis. As shown in FIG. 9B, the display 950 may include the diagnostic image (also referred to as “first readout”) 952 and a visualization 954 of the pressure measurement. In the diagnostic image 952, five dots corresponding to the cross-section of five linear segments can be visible. Based on the example of increments shown in FIG. 8, the example 910 may correspond to a measurement of 21 mm Hg, as provided in the visualization 954.

FIG. 10A shows another example 1010 of the gas-fluid changes in gas-fluid interface 340, 540 within the readout section 372, 392 based on the state of a target region of patient. In this example, the membrane/surface of the fluid reservoir 390, 590 caused the fluid 392, 592 to move through the six linear segments and five junction segments of the channel 350, 950, as shown in FIG. 10A. FIG. 10B shows an example 1050 of a diagnostic image (“also referred to as “first readout”) of the example 1010 acquired by an ultrasound transducer along the first readout axis 363, 563. As shown in FIG. 10B, two dots corresponding to the cross-section of two linear segments can be visible. Based on the example of increments shown in FIG. 8, the example 1010 may correspond to a measurement of 42 mm Hg.

In these examples, the pressure measurement may be determined based on the first readout. In some examples, the pressure measurement may be alternatively and/or additionally determined based on the readout(s) along the second readout axis.

In some examples, the pressure measurement may be based on the readouts along the second readout axis. In some examples, the diagnostic images (also referred to as “first readouts”) 952 or 1050 may be used to identify one or more linear segments for which an image (also referred to a “second readout”) should be taken along the second readout axis. For example, a second readout may be performed when the pressure determined from the first readout range is within a specific range of pressure measurements (e.g., not low or high pressure). In some examples, the first readout may determine a range of pressure measurements and the second readout(s) may determine a more precise pressure measurement.

In some examples, the second readout may be acquired and/or the quantitative measurement may be determined along the respective second readout axis for 1) a linear segment that has an invisible or dark dot next to a linear segment that has a visible dot; and/or 2) the linear segment that has a visible dot. By way of example, a second readout may be acquired for linear segments 912 and/or 914 shown in FIG. 9A. In some examples, the pressure measurement may be determined based on the one or more second readouts. In further examples, the pressure measurement may be determined based on both second readouts. For example, the pressure measurement may correspond to a total of the pressure measurements determined for the second readout(s) (e.g., for segments 912 and 914) acquired.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. An implantable pressure sensor device, comprising:
a fluid reservoir filled with a fluid;
a gas chamber filled with a gas; and
a gas-fluid interface including a channel;
the channel having a first end and a second end;
the channel having a pattern;
the pattern including a plurality of linear segments connected by a plurality of junction segments;
the fluid reservoir being in flow communication with the gas-fluid interface; and
the gas chamber being in flow communication with the gas-fluid interface and spaced separate from the gas-fluid interface.

2. The implantable pressure sensor device according to claim 1, further comprising:
one or more sets of imaging markers, each set of imaging markers defining a readout axis with respect to one or more of the plurality linear segments and/or of the plurality of junction segments of the gas-fluid interface, each readout axis corresponding to a quantitative measurement of pressure;
wherein the one or more sets of imaging markers and the gas-fluid interface define a readout section, the one or more sets of imaging markers being disposed on a boundary of the outreadout section;
the one or more sets of imaging markers including a first set of imaging markers, the first set of imaging markers defining a first readout axis that traverses each linear segment and having a first quantitative measurement of pressure, a cross-section of each linear segment along the first imaging axis corresponding to one increment of the first quantitative measurement of pressure.

3. The implantable pressure sensor device according to claim 2, wherein:
the one or more sets of imaging markers including a second set of imaging markers, the second set of imaging markers defining a second readout axis along one linear segment of the plurality of linear segments, a cross-section of the one linear segment along the second imaging axis corresponding to a second quantitative measurement of pressure.

4. The implantable pressure sensor device according to claim 3, wherein:
the fluid reservoir includes one or more surfaces configured to deflect based on pressure; and
when the fluid reservoir deflects, the fluid is configured to move from the fluid reservoir to the gas-fluid interface.

5. The implantable pressure sensor device according to claim 2, wherein:
the fluid reservoir is in flow communication with a first end of the gas-fluid interface; and
the gas chamber is in flow communication with the second end of the gas-fluid interface.

6. The implantable pressure sensor device according to claim 5, further comprising:
a first conduit connecting the fluid reservoir and the gas-fluid interface.

7. The implantable pressure sensor device according to claim 6, further comprising:
a second conduit connecting the gas chamber and the gas-fluid interface.

8. The implantable pressure sensor device according to claim 7, further comprising:
a device body, the device body including the readout section.

9. The implantable pressure sensor device according to claim 8, wherein the second conduit and the gas chamber are disposed external to the device body.

10. The implantable pressure sensor device according to claim 9, further comprising:
a testing member, the testing member including the gas chamber and an actuating member configured to engage a deflecting surface of the gas chamber.

11. The implantable pressure sensor device according to claim 8, wherein the fluid reservoir is disposed external to the device body.

12. The implantable pressure sensor device according to claim 11, wherein the fluid reservoir is a balloon.

13. The implantable pressure sensor device according to claim 2, wherein the device body includes one or layers, the one or more layers includes a first layer, the first layer including the readout section.

14. The implantable pressure sensor device according to claim 13, wherein the one or more layers include a first layer and third layer, the first layer being disposed between the second layer and the third layer, the third layer including the fluid reservoir and the second layer including the readout section.

15. The implantable pressure sensor device according to claim 14, wherein the gas chamber is disposed in the second layer and is separated and distinct from the readout section.

16. The implantable pressure sensor device according to claim 1, wherein:
a circumferential surface of the fluid reservoir is configured to deflect based on pressure; and
when the fluid reservoir deflects, the fluid is configured to move from the fluid reservoir to the gas-fluid interface.

17. The implantable pressure sensor according to claim 16, further comprising:
a device body, the device body including the gas-fluid-interface; and
a first conduit connecting the fluid reservoir and the gas-fluid interface;
wherein the first conduit and the fluid reservoir are disposed external to the device body.

18. The implantable pressure sensor device according to claim 17, further comprising:
one or more sets of imaging markers, each set of imaging markers defining a readout axis with respect to one or more segments of the gas-fluid interface, each readout axis corresponding to a quantitative measurement of pressure;
wherein the one or more sets of imaging markers and the gas-fluid interface define a readout section, the one or more sets of imaging markers being disposed on a boundary of the readout section, the device body including the readout section;
the one or more sets of imaging markers including a first set of imaging markers, the first set of imaging markers defining a first readout axis that traverses each linear segment and having a first quantitative measurement of pressure, a cross-section of each linear segment along the first imaging axis corresponding to one increment of the first quantitative measurement of pressure.

19. The implantable pressure sensor device according to claim 18, wherein:
the one or more sets of imaging markers including a second set of imaging markers, the second set of imaging markers defining a second readout axis along one linear segment of the plurality of linear segments, a cross-section of the one linear segment along the second imaging axis corresponding to a second quantitative measurement of pressure; and
the second readout axis is perpendicular to the first readout axis.

20. The implantable pressure sensor device according to claim 2, wherein the one or more sets of imaging markers are configured to be interrogated by an ultrasound.
